SEQUENCE LISTING (10 sequences)

SEQ ID NO: 1 - DNA, 24 bp, Artificial Sequence
  TB290 IS6110 outer forward
ggcgggacaacgccgaattgcgaa

SEQ ID NO: 2 - DNA, 24 bp, Artificial Sequence
  TB856 IS6110 outer reverse
cgagcgtaggcgtcggtgacaaag

SEQ ID NO: 3 - DNA, 20 bp, Artificial Sequence
  TB431 IS6110 inner forward
tactacgaccacatcaaccg

SEQ ID NO: 4 - DNA, 20 bp, Artificial Sequence
  TB740c IS6110 inner reverse
gggctgtggccggatcagcg

SEQ ID NO: 5 - DNA, 19 bp, Artificial Sequence
  IS6110-F IS6110 forward
ctcacggttcagggttagc

SEQ ID NO: 6 - DNA, 18 bp, Artificial Sequence
  IS6110-R IS6110 reverse
ctcaaggagcacatcagc

SEQ ID NO: 7 - DNA, 18 bp, Artificial Sequence
  esxA-F Rv3875 forward
gacagagcagcagtggaa

SEQ ID NO: 8 - DNA, 18 bp, Artificial Sequence
  esxA-R Rv3875 reverse
caaggagggaatgaatgg

SEQ ID NO: 9 - DNA, 18 bp, Artificial Sequence
  lpqG-F Rv3623 forward
ccgattggtccgtcattc

SEQ ID NO: 10 - DNA, 18 bp, Artificial Sequence
  lpqG-R Rv3623 reverse
gagcgatcccgagttgtg

(19) United States
(12) Patent Application Publication
(10) Pub. No.: US 2015/0152485 A1
(43) Pub. Date: Jun. 4, 2015

(54) MYCOBACTERIUM TUBERCULOSIS DETECTION USING TRANSRENAL DNA

(71) Applicant: THE JOHNS HOPKINS UNIVERSITY, Baltimore, MD (US)

(72) Inventors: Petros C. Karakousis, Towson, MD (US); Lee G. Klinkenberg, Pikesville, MD (US); Edith Torries-Chavolla, Baltimore, MD (US)

(73) Assignee: John Hopkins University, Baltimore, MD (US)

(21) Appl. No.: 14/408,592

(22) PCT Filed: Jun. 19, 2013
(86) PCT No.: PCT/US2013/046561

§ 371 Date: Dec. 17, 2014

Related U.S. Application Data
(60) Provisional application No. 61/661,566, filed on Jun. 19, 2012.
(60) Provisional application No. 61/735,595, filed on Dec. 11, 2012.

(51) Int. Cl. C12Q 1/68

(52) U.S. Cl. CPC C12Q 1/689; C12Q 2600/158

(57) ABSTRACT

The present invention relates to the field of Mycobacterium tuberculosis (Mtb). More specifically, the present invention provides methods for detecting Mtb using transrenal DNA. In one embodiment, a method for detecting Mycobacterium tuberculosis (Mtb) in a subject comprises the step of detecting Mtb transrenal DNA fragments in a urine sample obtained from the subject.

### CROSS-REFERENCE TO RELATED APPLICATIONS

This application claims the benefit of U.S. Provisional Application No. 61/661,566, filed Jun. 19, 2012, and U.S. Provisional Application No. 61/735,595, filed Dec. 11, 2012, which applications are incorporated herein by reference in their entireties.

### STATEMENT OF GOVERNMENTAL INTEREST

This invention was made with U.S. government support under grant no. NIH-R01-AI083125 and NIH-R01-HL106786. The U.S. government has certain rights in the invention.

### FIELD OF THE INVENTION

The present invention relates to the field of Mycobacterium tuberculosis (Mtb). More specifically, the present invention provides methods for detecting Mtb using transrenal DNA.

### INCORPORATION-BY-REFERENCE OF MATERIAL SUBMITTED ELECTRONICALLY

This application contains a sequence listing. It has been submitted electronically via EFS-Web as an ASCII text file entitled “P12063-03_ST25.txt.” The sequence listing is 2,275 bytes in size, and was created on Jun. 19, 2013. It is hereby incorporated by reference in its entirety.

### BACKGROUND OF THE INVENTION

Infection with Mycobacterium tuberculosis is one of the deadliest infections worldwide, with 1.7 million deaths in 2009 [1]. The gold standard of diagnosis is either bacterial culturing from sputum samples, which requires at least three weeks for growth, or acid-fast staining of bacilli, both of which require biosafety level 3 (BSL3) facilities. Since sputum samples can be difficult to obtain and are only relevant for tuberculosis of the lung, and acid-fast staining of bacilli is prone to failure in HIV co-infected individuals, recent efforts are being directed to developing tests using mycobacterial biomarkers. Currently available non-culture based diagnostic tests detect either antigens circulating in blood or genomic DNA isolated from bacilli in a sputum sample. The antigen based tests, which detect a variety of specific epitopes, have variable performance characteristics and require relatively sophisticated and expensive laboratory resources to perform. Sophistication of laboratory testing, and the need for blood draw, limit the application of the assays, such that screening can be performed only infrequently and require health care facilities for phlebotomy and sample processing. Furthermore, the World Health Organization has issued a warning against the use of inaccurate blood tests for active tuberculosis, describing them as “A substandard test with unreliable results” [2]. This is the first time WHO has issued an explicit “negative” policy recommendation against a practice that is widely used in tuberculosis care. Non-culture sputum based assays are rapid but have the same limitations as sputum culture assays. Development of an easy, non-culture, non-sputum based assay would allow for screening of patients with non-pulmonary tuberculosis, tuberculosis in HIV co-infection, and patients who are unable to produce a sputum sample.

### SUMMARY OF THE INVENTION

The present invention is based, at least in part, on the development of a urine-based assay which detects the presence of transrenal DNA fragments from the mycobacterial genome. Three optimal target sequences have been identified which allow the detection of TB infection using only single PCR reactions of short fragments (approximately 70 bp), targeting esxA, IS6110, and lpqG. These results have been validated using human urine spiked with sonicated Mycobacterium tuberculosis DNA. The present invention allows for for non-invasive testing of all patients regardless of the site of infection and regardless of the ability to produce a sputum sample.

In further embodiments, the three target DNA sequences are used as biomarkers in optical and electrochemical based-biosensor platforms for TB detection. The target sequences can also be used in isothermal amplification assays. Other transrenal DNA fragments from the Mtb genome can be used with the present invention.

Accordingly, the present invention provides methods for the detection of Mtb. In one embodiment, a method for detecting Mycobacterium tuberculosis (Mtb) in a subject comprises the step of detecting Mtb transrenal DNA fragments in a urine sample obtained from the subject. In certain embodiments, the detecting step is performed using polymerase chain reaction. In a specific embodiment, the Mtb transrenal DNA fragments comprise IS6110, esxA, and lpqG. In particular embodiments, the subject is a human. In another embodiment, the detecting step is performed using loop-mediated isothermal amplification.

In a further embodiment, a method for detecting Mtb in a patient comprises the steps of (a) providing a urine sample from the patient; and (b) performing an assay to detect the transrenal DNA fragments IS6110, esxA and lpqG in the sample, wherein detection of the fragments confirms the presence of Mtb in the patient. In certain embodiments, the assay is PCR amplification. In a specific embodiment, IS6110 is amplified using the primers shown in SEQ ID NO:1 and SEQ ID NO:2. In another specific embodiment, IS6110 is amplified using the primers shown in SEQ ID NO:3 and SEQ ID NO:4. In an alternative embodiment, IS6110 is amplified using the primers shown in SEQ ID NO:5 and SEQ ID NO:6. In another embodiment, esxA is amplified using the primers shown in SEQ ID NO:7 and SEQ ID NO:8. In yet another embodiment, lpqG is amplified using the primers shown in SEQ ID NO:9 and SEQ ID NO:10.

In a further embodiment, a method for diagnosing a patient as having Mtb comprises the step of detecting the presence of Mtb transrenal DNA fragments in the urine of the patient using polymerase chain reaction, wherein the detection provides the diagnosis.

In a specific embodiment, the Mtb transrenal DNA fragments comprise IS6110, esxA, and lpqG. In a more specific embodiment, IS6110 is amplified using the primers shown in SEQ ID NO:1 and SEQ ID NO:2. In another specific embodiment, IS6110 is amplified using the primers shown in SEQ ID NO:3 and SEQ ID NO:4. In an alternative embodiment, IS6110 is amplified using the primers shown in SEQ ID NO:5 and SEQ ID NO:6. In another embodiment, esxA is amplified using the primers shown in SEQ ID NO:7 and SEQ ID NO:8. In yet another embodiment, lpqG is amplified using the primers shown in SEQ ID NO:9 and SEQ ID NO:10.

### BRIEF DESCRIPTION OF THE FIGURES

FIG. 1. Primers Show Differing Specificity for Mycobacterium Tuberculosis Complex (MTC). Gel showing that M. bovis and M. bovis-BCG strains do not contain the targets sequence of lpqG and were negative by PCR. (A) M. tuberculosis; (B) M. bovis; (C) BCG. For (A) and (B): 1-Ladder; 2-IS6110; 3-esxA; 4-IpgG. For (C): 1-IS6110; 2-BCG; 3-IpqG; 4-Ladder.

FIG. 2. DNA can be PCR Amplified after Extraction from Human Urine.

FIG. 3. Sheared Mycobacterial Genomic DNA Passes from the Blood to the Urine.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Infection with Mycobacterium tuberculosis (Mtb) is one of the deadliest infections worldwide, causing 2 million deaths annually. The diagnostic gold standard is sputum bacterial culturing, which requires at least three weeks for growth. Since sputum samples are often unavailable and relevant only for pulmonary tuberculosis, and sputum smear microscopy lacks sensitivity in HIV-infected individuals, recent efforts have focused on identifying novel diagnostic biomarkers. The present inventors have developed a urine-based assay which detects the presence of transrenal DNA fragments from the mycobacterial genome, allowing for non-invasive testing of all patients regardless of the site of infection and regardless of the ability to produce a sputum sample. Nested PCR analysis shows that Mtb genomic DNA (MGD), fragmented by sonication, can be isolated and detected when spiked into human urine. Fragmented MGD was then injected into guinea pigs via jugular vein catheter. Detection was possible from the urine sample collected spanning the first six hours after injection and more easily from an overnight sample. Urine was also collected from guinea pigs at day 21 and day 28 after aerosol infection with ˜100 bacilli/lung of Mtb CDC1551. PCR analysis successfully detected MGD in these samples. In a mouse aerosol model, several successive urine samples were collected after infection with Mtb. Starting at day 21 after infection, urine was collected daily and pooled into weekly samples. After week 4, the mice were treated daily with 25 mg/kg/day isoniazid and samples from week 5 and 6 were collected. All four samples tested positive for MGD, but most strongly in the 3rd and 4th week samples. Finally, optimal sequences were identified which allow the detection of infection using only single PCR reactions of short fragments (approximately 70 bp), targeting esxA, IS6110, and lpqG. These results have been validated using human urine spiked with sonicated MGD. Urine-based assays to detect MGD provide a rapid, reliable, and noninvasive method to detect Mtb infection.

Mycobacterial antigens have been shown to be present in the urine of infected patients [3]. The present inventors endeavored to identify mycobacterial genomic DNA, in the absence of intact bacilli, in the urine of infected animals. The validation of an animal model for trans-renal DNA detection allowed the present inventors to carefully test limits of detection, detection at different time points of infection, and detection changes in response to different therapies. It also provided a reproducible standard which allows direct comparison of current and future technologies of DNA isolation and detection.

Amplicon: A term for any relatively small, DNA fragment that is replicated, e.g., by PCR.

Amplification: An increase in the number of copies of a specific DNA fragment can occur in vivo or in vitro.

Gene: DNA fragment that contains sequences necessary to code for an mRNA, and to control the expression of these sequences.

Genome: The total set of genes of an organism enclosed, among the eukaryotes, in chromosomal structures.

Hybridization: A widely used technique that exploits the ability of complementary sequences in single-stranded DNAs or RNAs to pair with each other to form a double helix. Hybridization can take place between two complimentary DNA sequences, between a single-stranded DNA and a complementary RNA, or between two RNA sequences. The technique is used to detect and isolate specific sequences, measure homology, or define other characteristics of one or both strands.

Nested PCR: A second PCR that is performed on the product of an earlier PCR using primer, which are internal to the originals. This significantly improves the sensitivity and specificity of the PCR.

Nested primer: A selected primer internal to an amplicon obtained with a first PCR cycle. The amplification process that uses at least one nested primer improves specificity, because the non-specific products of the first cycle are not amplified in the second cycle.

Nucleic Acid: Linear polymers of nucleotides, linked by 3′, 5′ phosphodiester linkages. In DNA, deoxyribonucleic acid, the sugar group is deoxyribose and the bases of the nucleotides adenine, guanine, thymine and cytosine. RNA, ribonucleic acid, has ribose as the sugar and uracil replaces thymine DNA functions as a stable repository of genetic information in the form of base sequence. RNA has a similar function in some mycobacteria but more usually serves as an informational intermediate (mRNA), a transporter of amino acids (tRNA), in a structural capacity or, in some newly discovered instances, as an enzyme.

Oligonucleotide/Polynucleotide: Linear sequence of two or more nucleotides joined by phosphodiester bonds. Above a length of about 20 nucleotides the term “polynucleotide” is generally used.

Polymerase: Enzyme utilized in the amplification of nucleic acids. The term includes all of the variants of DNA polymerases.

Primer: Short pre-existing polynucleotide chain to which new deoxyribonucleotides can be added by DNA polymerase.

PCR: Polymerase Chain Reaction involving two synthetic oligonucleotide primers, which are complementary to two regions of the target DNA (one for each strand) to be amplified, are added to the target DNA (that need not be pure), in the presence of excess deoxynucleotides and Taq polymerase, a heat stable DNA polymerase. In a series (typically 30) of temperature cycles, the target DNA is repeatedly denatured (around 90° C.), annealed to the primers (typically at 50-60° C.) and a daughter strand extended from the primers (72° C.). As the daughter strands themselves act as templates for subsequent cycles, DNA fragments matching both primers are amplified exponentially, rather than linearly.

Probe: General term for a fragment of DNA or RNA corresponding to a gene or sequence of interest that has been labelled either radioactively or with some other detectable molecule, such as biotin, digoxygenin or fluorescein.

Sample: The term is broadly interpreted and includes any form that contains nucleic acids (DNA or RNA) in solution or attached to a solid substrate, where the definition of “nucleic acids” includes genomic DNA (for example, when it is attached to a solid substrate, such as in the Southern Blot or in solution), cDNA, and other forms.

Combinations of two nucleic-acid sequences through hybridization are formed thanks to the hydrogen bonds between G and C or A and T bases or analogs of these bases. These combinations are complementary, and the DNA helixes are anti-parallel. This hybridization combination can be created with one sequence (or helix) in a solution and the other attached to a solid phase (such as, for example, in the FISH [fluorescent in situ hybridization]method), or else with both of the sequences in solution.

Target sequence: Nucleic-acid sequence that should be analyzed through hybridization, amplification, or other methods or combinations of methods.

Tm (melting temperature): Temperature at which a specific double-helix DNA population dissociates into single-strand polymers. The formula for calculating this temperature for polynucleotide fragments is well known in the art: Tm=81.5+0.41(% G+C) (Anderson & Young, “Quantitative Filter Hybridization,” in Nucleic Acid Hybridization [1985]). For oligonucleotides with fewer than 40 base pairs, a simplified formula can be used: Tm=3° C.×(G+C)+2×(A+T).

Tr-DNA/RNA: Transrenal DNA/RNA, or DNA/RNA present in urine after having been passed through the kidney barrier.

Urinary tract: Includes the organs and ducts that participate in the elimination of urine from the body.

Urinary Nucleic Acids in Mycobacterial Pathogen Infections

The present invention is based on the discovery that following a mycobacterial infection, the nucleic acids of the bacteria are cleaved into relatively short fragments which are found in the urine. Many of these bacterial specific nucleic acids cross the transrenal barrier (these nucleic acids are generally termed TrNA, or TrDNA or TrRNA) and can be detected in urine as cell-free low-molecular-weight fragments (whose length is less than 1000 nucleotides, but are preferably less than 500 bp in length, and more preferably shorter than 250-300 bp in length or shorter than 250 bp in length) through molecular methods. These transrenal nucleic acids are derived from mycobacteria which are located outside of the urinary tract of a subject. As used herein, the term “mycobacterial nucleic acid” encompasses nucleic acids of mycobacterial origin. Other mycobacteria specific nucleic acids may be shed by mycobacteria or cells that are within the kidney, and thus do not have to cross the transrenal barrier in order to be detected in the urine. Further, some mycobacteria specific nucleic acids may be found in the urine through other mechanisms besides crossing the transrenal barrier or being generated by mycobacteria in the kidney.

The presence of transrenal nucleic acids of mycobacterial origin in the case of mycobacterial infections according to the present invention is also, and preferably, detected in the case of non-urinary-tract infections, even in the absence of hematuria or of pathologies that lead to the rupture, or that alter the normal integrity, of the renal barrier.

Transrenal nucleic acids (Tr-NA) of mycobacterial origin are not associated with, and are not derived from, the genome of mycobacteria that are lost or released in the urinary tract and that are found in urine. Instead, transrenal nucleic acids are filtered by the glomerular-renal filtration mechanism. Thus, the dimensions of the transrenal nucleic-acid fragments are generally smaller than about 1000 base pairs, e.g., smaller than about 500, smaller than about 300, smaller than about 250, or between about 100 and about 200 base pairs, as opposed to other situations in which DNA usually has a high molecular weight and a length in excess of 1000 bases or base pairs.

Therefore, in the present invention, the transrenal nucleic acid (TrNA) of mycobacterial origin is generally not found in the urine sediment, but in the soluble fraction, although traces of TrNA can co-sediment with the cells during centrifuging.

The discovery confirms the presence of urinary nucleic acids or transrenal nucleic acids derived from mycobacteria in urine, and therefore is applicable to the diagnosis of all infectious diseases caused by mycobacterial pathogens.

Therefore, in embodiments, the invention relates to methods for diagnosis or monitoring of mycobacterial infection by determining the presence of mycobacterial nucleic acids, preferably mycobacterial DNA or RNA of mycobacterial origin, in a urine sample. The methods includes the step of determining the presence of transrenal mycobacterial nucleic acids using methods generally used in laboratory practice such as hybridization, PCR, nested PCR, semi-nested PCR, real-time PCR, quantitative PCR, and the like.

In certain embodiments, the methods according to the invention include an initial treatment of the urine sample prior to the determination of the presence of transrenal mycobacterial nucleic acids. In an embodiment, the invention includes the pretreatment of the urine sample with an agent that inhibits the degradation of the DNA or RNA. These agents include the enzymatic inhibitors, such as chelating agents, detergents, or denaturing agents, DNase or RNase inhibitors, which are preferably selected from the group consisting of EDTA, guanidine HCl, guanidine isothiocyanate, N-lauryl sarcosine, and sodium dodecyl sulfate.

In another embodiment, the determination of the presence of transrenal mycobacterial nucleic acids optionally is preceded by centrifugation or filtration of the urine sample in order to separate the cellular fraction of the urine from the cell-free low-molecular-weight nucleic acids (DNA/RNA). However, the urine sample may also be utilized without fractionation. Centrifugation can be performed at a speed between about 2500 g and about 4500 g, between about 3000 g and about 4000 g. Filtration is preferred to carry out through a filter with pore size between about 0.1 and about 5.0 μm, between about 0.2 and about 1.0 μm and about 0.45 and 0.8 about μm. Equivalent methods for separating the soluble fraction from the cellular fraction may also be used.

The optional isolation and/or purification of the transrenal nucleic acids can be achieved through the use of chemical or physical methods that are already known in the art. It includes one or more purification steps using methods selected from among extraction with organic solvents, filtration, precipitation, absorption on solid matrices (e.g., silica resin, hydroxyapatite or ion exchange), affinity chromatography (e.g., via sequence specific capture or nucleic acid specific ligands), or else molecular exclusion chromatography. However, the purification method must be appropriate for the isolation of DNA (single- or double-strand) whose dimensions are smaller than about 1000 nucleotide pairs, smaller than about 500 nucleotides, and fragments whose length are less than about 300 or about 250 base pairs, or that are between about 100 and about 200 bases or base pairs. The purification can take place on a matrix including, but not limited to, silica resin.

In one embodiment, the DNA isolation method is implemented by pretreating the urine sample with a denaturing agent, as described above, e.g., urea, guanidine HCl, or guanidine isothiocyanate, at room temperature. Guanidine isothiocyanate is preferably utilized. The sample is then passed through a solid phase, preferably a matrix consisting of a silica resin that, in the presence of chaotropic salts (guanidine isothiocyanate), binds the nucleic acids. The sample is then collected or eluted in a buffer, such as Tris-EDTA (Tris 10 mM, EDTA 1 mM), or in water.

In another preferred embodiment, the characterization and the determination of the presence of transrenal mycobacterial DNA are performed through techniques including, but not limited to, hybridization of the nucleic acids, a cycling probe reaction), a polymerase chain reaction (PCR Protocols: A Guide to Methods and Applications, by M. Innis et ah; Elsevier Publications, 1990), a nested polymerase chain reaction, single-strand conformation polymorphism, a ligase chain reaction (LCR) (F. Barany, in PNAS USA, 88:189-93 [1991]), strand displacement amplification (SDA) (G. K. Terrance Walker, et ah, in Nucleic Acid Res, 22:2670-77 [1994], and restriction fragments length polymorphism (RFLP). A technician in the field might also use combinations of these methods, e.g., PCR-Restriction Length Polymorphism, in which the nucleic acids are amplified, and then divided into aliquots and digested with restriction enzymes, and then separated via electrophoresis.

In particular embodiments, polymerase chain reaction (PCR) is the preferred method for the detection and/or quantitative analysis of nucleic acids. In other embodiments, nested PCR is used, as defined above, or the semi-nested PCR method, in which only one of the two primers is internal to the amplicon.

The advantage of the method is linked primarily to the ease of collecting the biological samples; to the fact that the transrenal nucleic acids are not infectious; and to the sensitivity of the molecular diagnostic method that can be applied to the nucleic acids, even in the form of fragments.

In another of its embodiments, the invention relates to a kit for the detection and monitoring of transrenal mycobacterial DNA in urine, including: reagents and/or materials for the separation and/or purification of transrenal DNA from a urine sample, DNA probes, or pairs of specific oligonucleotides (primers) for at least one mycobacterial agent. Reaction tubes, agents for the pretreatment of the sample, enzymes for labeling the probe, and enzymes for the amplification of the DNA may optionally be present. In a preferred embodiment, the kit includes pairs of oligonucleotide primers that are specific for mycobacteria. The kit may specifically comprise primers that are selected from the group consisting of the sequences listed below, and specific reagents for the polymerization chain reaction.

Methods for the Amplification and Detection of Urinary Nucleic Acids

The term nucleic acid refers to an oligonucleotide, nucleotide, polynucleotide, or fragments/parts thereof and to DNA or RNA of natural (e.g., genomic) or synthetic origin. It may have a double or single helix, and may also represent the sense or antisense direction of a sequence. The terms oligonucleotide, polynucleotide and nucleic-acid polymer are equivalent, and are understood as referring to a molecule consisting of more than two deoxyribonucleic or ribonucleic acid bases. The number of nucleotides (bases) and the length of the oligonucleotide fragment may vary. They may be synthesized in different ways. The sequences are traditionally defined as starting with 5′ and ending with a 3′. These numbers indicate the direction of the sequence.

DNA isolated from the urine of a subject may then be amplified in order to be detected.

Amplification methods include polymerase chain reaction (PCR), nested PCR, semi-nested PCR, Single-Strand Conformation Polymorphism analysis (SSCP), ligase chain reaction (LCR) and strand displacement amplification (SDA). Detection of transrenal DNAs is also performed through hybridization of at least one labeled primer.

Hybridization is a method that allows two nucleic-acid sequences to recognize each other as complementary and to join together (annealing). Complementarity/Complementary sequences are sequences of polynucleotides that interact with each other, depending on the interaction between the bases. For example, the AGTC sequence is complementary to TCAG according to standard Watson Crick base pairing. However, other combinations such as Hoogstein base pairing are well known to those having ordinary skill in the art. It is possible to have a fully or partially complementary sequence, and this is what determines the efficiency or attractive force between the two sequences. Average complementarity would prevent a strong complementarity from hybridizing, under conditions that would allow it to remain attached.

The ability of nucleic sequences to hybridize is a well-known phenomenon. The first hybridization method was described in Marmur & Lane, PNAS USA, 46:453 (1960) and 461 (1960), but since then has been perfected as a technique in molecular biology. Today, the term “hybridization” includes, among others, slot/dot and blot hybridization. The conditions that allow nucleotide sequences to recognize each other (hybridization) can be modified in such a way as to produce complete hybridization (complementarity with high specificity) or partial hybridization (complementarity with average specificity). In the present application, whenever the term “hybridization” is used, the conditions should be understood as referring to those that allow average or high complementarity. The technician in the field can calculate how many artificial sequences are needed to encourage hybridization between two complementary sequences in the opposite direction, known as antiparallel association.

A probe is an oligonucleotide that can be produced artificially or naturally, and that forms a combination with another nucleic-acid sequence. The probes are useful in discovering specific sequences in a sample containing unknown DNA. In some embodiments, all of the probes can be bound to a signaling molecule (or reporter). The reporter molecule makes it possible to detect the probe (for example, through enzymatic reactions (e.g., ELISA (Enzyme-Linked Immunosorbent Assay)), radioactivity, fluorescence, or other systems).

Polymerase chain reaction (PCR) is a method of amplification of a DNA sequence using complementary primers and a heat sensitive polymerase. One class of enzymes utilized in the amplification of specific nucleic acids are DNA polymerases referred to as Taq (Thermus aquations) polymerases. Primers are oligonucleotides from which, under proper conditions, the synthesis of a polynucleotide sequence can be initiated. A primer may exist naturally (for example, in an enzymatic digestion of a polynucleotide), or may be obtained through chemical synthesis. The product amplified in PCR is often referred to as an amplicon.

Nested PCR is a second PCR which is performed on the product of an earlier PCR using a second set of primers which are internal to the first set of primers, referred to as nested primers. This significantly improves the sensitivity and specificity of the PCR. Nested primers are primers internal to an amplicon obtained with a first PCR cycle. The amplification process that uses at least one nested primer improves specificity, because the non-specific products of the first cycle are not amplified in the second cycle, because they lack the sequence that corresponds to the nested primer. Semi-nested PCR is a second PCR which uses one new primer and one of the original primers. This process also improves specificity.

Ligase Chain Reaction (LCR) is a method of DNA amplification similar to PCR. LCR differs from PCR because it amplifies the probe molecule rather than producing an amplicon through polymerization of nucleotides. Two probes are used per each DNA strand and are ligated together to form a single probe. LCR uses both a DNA polymerase enzyme and a DNA ligase enzyme to drive the reaction. Like PCR, LCR requires a thermal cycler to drive the reaction and each cycle results in a doubling of the target nucleic acid molecule. LCR can have greater specificity than PCR.

In Single-Strand Conformation Polymorphism (SSCP) analysis a small PCR product (amplicon) is denatured and electrophoresed through a non-denaturing polyacrylamide gel. Thus, as the PCR product moves into and through the gel (and away from the denaturant), it will regain secondary structure that is sequence dependent (similar to RNA secondary structure). The mobility of the single-stranded PCR products will depend upon their secondary structure.

Therefore, PCR products that contain substitutional sequence differences as well as insertions and deletions will have different mobilities.

Strand displacement amplification (SDA) is an isothermal nucleic acid amplification method based on the primer-directed nicking activity of a restriction enzyme and the strand displacement activity of an exonuclease-deficient polymerase.

The terms purification or isolation refers to a process for removing contaminants from a sample, where the result is a sample containing about 50%, about 60%, about 75%, about 90% or over about 90% of the material toward which the purification procedure is directed.

For stringent temperature conditions in the case of nucleic-acid hybridization, these terms usually refer to a variable temperature between a maximum, for a nucleic acid, represented by Tm less about 5° C., and a minimum represented by Tm less about 25° C. The technique used in the field utilizes stringent temperature conditions, in combination with other parameters (e.g., saline concentration), to distinguish sequences with a quasi-exact homology.

Stringent conditions are known to those skilled in the art and can be found in Ausubel, et ah, (eds.), CURRENT PROTOCOLS IN MOLECULAR BIOLOGY, John Wiley & Sons, N.Y. (1989), 6.3.1-6.3.6. Preferably, the conditions are such that sequences at least about 65%, about 70%, about 75%, about 85%, about 90%, about 95%, about 98%, or about 99% homologous to each other typically remain hybridized to each other. A non-limiting example of stringent hybridization conditions are hybridization in a high salt buffer comprising 6×SSC, 50 niM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C.

In another embodiment, a nucleic acid sequence that is hybridizable to the nucleic acid molecule comprising the nucleotide sequence, or fragments, analogs or derivatives thereof, under conditions of moderate stringency is provided. A non-limiting example of moderate stringency hybridization conditions are hybridization in 6×SSC, 5× Reinhardt's solution, 0.5% SDS and 100 mg/ml denatured salmon sperm DNA at 55° C., followed by one or more washes in IX SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, NY.

In yet another embodiment, a nucleic acid that is hybridizable to the nucleic acid molecule, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 niM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

The invention also provides a kit for detecting and/or genotyping a mycobacterial nucleic acid in a urine sample from a subject in need thereof, comprising at least one forward primer including those described herein and at least one reverse primer including those described herein either in the same or separate packaging, and instructions for its use. In one embodiment, this mycobacterial nucleic acid is derived from mycobacterium tuberculosis.

The invention will be further described in the following examples, which do not limit the scope of the invention described in the claims.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Bacterial Strains, Media, and Growth Conditions.

The H37Rv and CDC1551 strains of M. tuberculosis were grown with shaking to mid-logarithmic phase (OD600˜0.6) at 37° C. in Middlebrook 7H9 liquid broth (Difco) supplemented with oleic acid-albumin-dextrose-catalase (Becton Dickinson) and either 0.05% Tween-80 or 0.05% tyloxapol.

Preparation of Fragmented Genomic DNA.

M. tuberculosis CDC1551 genomic DNA was purified as previously described [4] and treated with 6 cycles of sonication, 30 seconds at a power setting of six, followed by 1 minute on ice, using a Model 100 Sonic Dismembrator (Fisher) with a ⅛″ probe. The fragment size of the sonicated DNA was determined from a sample run on a 2.5% TAE agarose gel to be between 100-400 bp in length (data not shown).

Isolation of Genomic DNA from Urine Samples.

Urine samples were collected, stored briefly at room temperature and then frozen at −80° C. for up to several weeks. The soluble fraction of the thawed urine was collected by centrifugation of a 6 mL sample for 10 min, at 1500×g, and 4° C. The solution was then added to MaXtract High Density 15 mL columns (Qiagen) along with an equal volume of 25:24:1 phenol:chloroform:isoamyl alcohol, equilibrated with Tris pH 8.0. Samples were mixed and then separated with a 2 min, 1500×g, 4° C. spin. The aqueous layer was collected by decanting and the MaX tract column extraction was repeated 2 more times. The column extraction was then repeated once with an equal volume of 24:1 chloroform:isoamyl alcohol. The final aqueous layer was stored overnight at −20° C. after the addition of 0.25 volumes of 10M ammonium acetate and 2.5 volumes of 100% ethanol. The DNA samples were collected by centrifugation at 20,000×g for 20 minutes at 4° C. The pellet was washed with 70% ethanol, and air dried for at least 20 minutes. The pellets were resuspended in 100-300 uL Dnase/Rnase free H₂O and the supernatant was collected at 12,000×g, for 2 minutes. The DNA samples were stored at −20° C. until used. Qiagen columns.

PCR Amplification of Mycobacterial Specific Sequences.

PCR amplification was performed using primer pairs shown in Table 1. Samples were subjected to 35 cycles of amplification, 1 minute at 94° C., 45 seconds at 55° C., and 30 seconds at 72° C., using platinum Taq polymerase (Invitrogen) in a 25 μL reaction volume. Samples were then loaded onto a 2.5% agarose gel and visualized with ethidium bromide staining. Nested PCR reactions used 3 μL of primary (outer primers) PCR as template for second round (inner primers) PCR.

TABLE 1
Primers Used in This Study

| Primer | Descrip- | | |
|---|---|---|---|
| Designation | tion | Sequence | Source |
| TB290 | IS6110 | GGCGGGACAACGCCGAATTG | [6, 7] |
| | outer | CGAA (SEQ ID NO: 1) | |
| | forward | | |
| TB856 | IS6110 | CGAGCGTAGGCGTCGGTGAC | [6, 7] |
| | outer | AAAG (SEQ ID NO: 2) | |
| | reverse | | |
| TB431 | IS6110 | TACTACGACCACATCAACCG | [6, 7] |
| | inner | (SEQ ID NO: 3) | |
| | forward | | |
| TB740c | IS6110 | GGGCTGTGGCCGGATCAGCG | [7] |
| | inner | (SEQ ID NO: 4) | |
| | reverse | | |
| IS6110-F | IS6110 | CTCACGGTTCAGGGTTAGC | This |
| | forward | (SEQ ID NO: 5) | Study |
| IS6110-R | IS6110 | CTCAAGGAGCACATCAGC | This |
| | reverse | (SEQ ID NO: 6) | Study |
| esxA-F | Rv3875 | GACAGAGCAGCAGTGGAA | This |
| | forward | (SEQ ID NO: 7) | Study |
| esxA-R | Rv3875 | CAAGGAGGGAATGAATGG | This |
| | reverse | (SEQ ID NO: 8) | Study |
| 1pqG-F | Rv3623 | CCGATTGGTCCGTCATTC | This |
| | forward | (SEQ ID NO: 9) | Study |
| 1pqG-R | Rv3623 | GAGCGATCCCGAGTTGTG | This |
| | reverse | (SEQ ID NO: 10) | Study |

Animal Urine Collection.

Animals were housed in a metabolic cage (Tecniplast), which allows for passive collection of urine, in isolation from other debris. Up to six mice or one guinea pig were housed in a single chamber to collect a sample. Samples were frozen at −80° C. and daily mouse samples were subsequently pooled to form weekly samples.

Guinea Pigs.

Hartley guinea pigs containing a jugular vein catheter (250-300 g, Charles River) were housed in a Biosafety Level-3 (BSL-3), specific pathogen-free animal facility and were fed water and chow ad libitum. The animals were maintained and all procedures performed according to protocols approved by the Institutional Animal Care and Use Committee at The Johns Hopkins University School of Medicine. Fragmented genomic DNA was injected into the jugular vein catheter, followed with 1 mL PBS to ensure entry of the DNA into the bloodstream. Two samples of urine were then collected, during the first 6 hours following injection and during the next 18 hours following injection.

Mice.

Four to six week-old female Balb/c mice were purchased from Charles River Labs (Wilmington, Mass.) Animals were housed in a BSL-3 facility, maintained under specific pathogen-free conditions, and fed water and chow ad libitum. All procedures followed protocols approved by the Institutional Animal Care and Use Committee at The Johns Hopkins University.

Infection of Animals.

Five mice were aerosol-infected with wild-type M. tuberculosis H37Rv using an inhalation exposure system (Glas-col) calibrated to deliver ˜1000 bacilli per animal. After 28 days of infection, the mice were treated with 25 mg/kg/day isoniazid by gavage with an esophageal cannula for two weeks and subsequently sacrificed. Weekly urine samples were collected for weeks 3 and 4 (prior to isoniazid treatment) and weeks 5 and 6 (during treatment). At the time of sacrifice, lungs, kidneys and bladder were separately homogenized for colony-forming unit (CFU) enumeration in 2 ml PBS using a Tenbroeck 2 mL tissue grinder (Kimble Chase). Serial ten-fold dilutions of organ homogenates were plated on 7H11 selective agar (BBL). Plates were incubated at 37° C. and CFU were counted after four weeks.

Primers Show Differing Specificity for Mycobacterium Tuberculosis Complex (MTC).

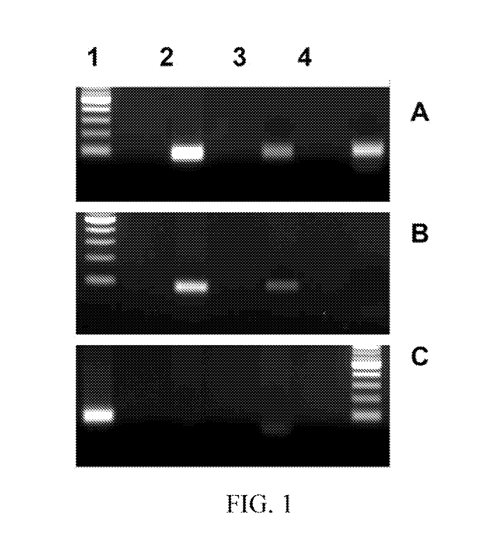

Using PCR amplification, the presence of the three target regions (IS6110, esxA, and lpqG) was tested for in several species of mycobacteria. See FIG. 1. It was confirmed that none of them were present in M. smegmatis, M. avium and M. marinum genomic DNA. Three strains from the MTC complex were evaluated: M. tuberculosis, M. bovis and M. bovis-BCG. M. tuberculosis tested positive for all the targets. The target IS6110, which is MTC specific, tested positive for all three strains. M. bovis and M. bovis-BCG strains do not contain the targeted sequence of lpqG and were negative by PCR. M bovis-BCG lacks RD1 which contains ESAT-6, esxA, so while Ill Bovis was positive for esxA amplification, the vaccine strain M bovis-BCG was negative. Templates purified from uninfected human, mouse, guinea pig, and rabbit urine samples also produced negative results with all of the primer pairs. All of these results were as one would predict based on a comparisons between the primer sequences and the genomic sequences of the species tested.

DNA can be PCR Amplified after Extraction from Human Urine.

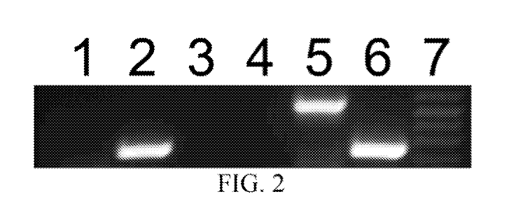

Human urine was collected from a tuberculin DTH skin test negative donor. One six milliliter aliquot was spiked with 12.5 μg of sheared M. tuberculosis genomic DNA dissolved in Tris-EDTA pH 8.0, and a negative-control sample was spiked with an equal volume of Tris-EDTA pH 8.0. A 6 ml volume of Tris-EDTA pH 8.0 was also spiked with 12.5 μg of sheared M. tuberculosis genomic DNA dissolved in Tris-EDTA pH 8.0, as a positive control. After purification the DNA sample pellet was resuspended in 48 μl of Tris-EDTA pH 8.0. From this sample, 0.3 μL were used as template to amplify IS6110, in a 25 uL reaction, using the primers TB290 and TB856. FIG. 2, lanes 1, 3 and 5. Each PCR reaction uses the DNA isolated from 37.5 μl of human urine containing 78.125 ng of spiked DNA, prior to purification. Three microliters of this PCR product was then used in a nested PCR reaction with primers TB431 and TB740c. FIG. 2, lanes 2, 4 and 6. Bands of the expected size were detected for both the primary PCR and the nested PCR, when the template was purified from Tris-EDTA pH 8.0 and a band of the expected size was detected in the nested PCR when the template was purified from the urine sample. The negative control samples did not produce PCR products.

Sheared Mycobacterial Genomic DNA Passes from the Blood to the Urine.

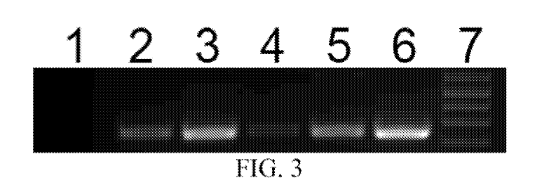

Two guinea pigs were injected through a jugular vein catheter with 10 and 30 μg of sonicated MGD, respectively. Urine collection commenced immediately following injection with the first set of samples frozen after 6 hours. After a subsequent 18 hours, a second and final sample was collected. DNA isolation was performed as above. Nested PCR with primers, TB290 and TB856, followed by primers, TB431 and TB740c was able to detect mycobacterial DNA in all four samples (FIG. 3, lanes 2-5), but not in a sample of urine collected from an untreated guinea pig (FIG. 3, lane 1). DNA added to 6 ml of TE, again, served as a positive control (FIG. 3, lane 6).

### REFERENCES

1. WHO global tuberculosis control report 2010. Summary Cent Eur J Public Health 2010; 18:237.
2. WHO. WHO warns against the use of inaccurate blood tests for active tuberculosis. A substandard test with unreliable results. Accessed Jul. 20, 2011.
3. Kashino 55, Pollock N, Napolitano D R, Rodrigues V, Jr., Campos-Neto A. Identification and characterization of Mycobacterium tuberculosis antigens in urine of patients with active pulmonary tuberculosis: an innovative and alternative approach of antigen discovery of useful microbial molecules. Clin Exp Immunol 2008; 153:56-62.
4. Ausubel F M. Current protocols in molecular biology. New York: Greene Pub. Associates and Wiley Interscience: J. Wiley, 1991.
5. Lurie MB. Resistance to tuberculosis; experimental studies in native and acquired defensive mechanisms. Cambridge, Published for the Commonwealth Fund by Harvard University Press, 1964.
6. Aceti A, Zanetti 5, Mura M S, et al. Identification of HIV patients with active pulmonary tuberculosis using urine based polymerase chain reaction assay. Thorax 1999; 54:145-6.
7. Torrea G, Van de Perre P, Ouedraogo M, et al. PCR-based detection of the Mycobacterium tuberculosis complex in urine of HIV-infected and uninfected pulmonary and extra pulmonary tuberculosis patients in Burkina Fa so. J Med Microbial 2005; 54:39-44.

1. A method for detecting Mycobacterium tuberculosis (Mtb) in a subject comprising the step of detecting Mtb transrenal DNA fragments in a urine sample obtained from the subject.

2. The method of claim 1, wherein the detecting step is performed using polymerase chain reaction.

3. The method of claim 1, wherein the Mtb transrenal DNA fragments comprise IS6110, esxA, and lpqG.

4. The method of claim 1, wherein the subject is a human.

5. The method of claim 1, wherein the detecting step is performed using loop-mediated isothermal amplification.

6. A method for detecting Mtb in a patient comprising the steps of:
a. providing a urine sample from the patient;
b. performing an assay to detect the transrenal DNA fragments IS6110, esxA and lpqG in the sample, wherein detection of the fragments confirms the presence of Mtb in the patient.

7. The method of claim 6, wherein the assay is PCR amplification.

8. The method of claim 7, wherein IS6110 is amplified using the primers shown in SEQ ID NO:1 and SEQ ID NO:2.

9. The method of claim 7, wherein IS6110 is amplified using the primers shown in SEQ ID NO:3 and SEQ ID NO:4.

10. The method of claim 7, wherein IS6110 is amplified using the primers shown in SEQ ID NO:5 and SEQ ID NO:6.

11. The method of claim 7, wherein esxA is amplified using the primers shown in SEQ ID NO:7 and SEQ ID NO:8.

12. The method of claim 7, wherein lpqG is amplified using the primers shown in SEQ ID NO:9 and SEQ ID NO:10.

13. A method for diagnosing a patient as having Mtb comprising the step of detecting the presence of Mtb transrenal DNA fragments in the urine of the patient using polymerase chain reaction, wherein the detection provides the diagnosis.

14. The method of claim 13, wherein the Mtb transrenal DNA fragments comprise IS6110, esxA, and lpqG.

15. The method of claim 14, wherein IS6110 is amplified using the primers shown in SEQ ID NO:1 and SEQ ID NO:2.

16. The method of claim 14, wherein IS6110 is amplified using the primers shown in SEQ ID NO:3 and SEQ ID NO:4.

17. The method of claim 14, wherein IS6110 is amplified using the primers shown in SEQ ID NO:5 and SEQ ID NO:6.

18. The method of claim 14, wherein esxA is amplified using the primers shown in SEQ ID NO:7 and SEQ ID NO:8.

19. The method of claim 14, wherein lpqG is amplified using the primers shown in SEQ ID NO:9 and SEQ ID NO:10.
